(19) United States
(12) Patent Application Publication
(10) Pub. No.: US 2025/0213482 A1
(43) Pub. Date: Jul. 3, 2025

(54) OXYGEN-ENHANCED ADAPTIVE FUNCTIONAL BEVERAGE FOR ANTI-AGING AND METHOD OF PREPARATION

(71) Applicant: Xu Fen Tu, San Gabriel, CA (US)

(72) Inventor: Xu Fen Tu, San Gabriel, CA (US)

(21) Appl. No.: 19/086,165

(22) Filed: Mar. 21, 2025

(51) Int. Cl. A61K 9/1277; A61K 31/05; A61K 31/122; A61K 31/197; A61K 31/405; A61K 31/4745; A61K 31/685; A61K 31/706; A61K 31/7084; A61K 36/41; A61K 36/63; A61K 45/06

(52) U.S. Cl. CPC A61K 9/1277; A61K 31/05; A61K 31/122; A61K 31/197; A61K 31/405; A61K 31/4745; A61K 31/685; A61K 31/706; A61K 31/7084; A61K 36/41; A61K 36/63; A61K 45/06

(57) ABSTRACT

A high-oxygen adaptive anti-aging oxygen-enhanced adaptive functional beverage formulated using advanced liposomal encapsulation, plant-based microencapsulation, and microbubble oxygenation technologies to enhance bioavailability, stability, and oxygen delivery. The oxygen-enhanced adaptive functional beverage improves cellular metabolism, mitochondrial function, and oxygen utilization, leading to increased energy levels, cognitive function, and oxidative stress resistance. By promoting efficient oxygen absorption and retention, the oxygen-enhanced adaptive functional beverage supports healthy aging, athletic performance, and high-altitude adaptation. The oxygen-enhanced adaptive functional beverage incorporates bioactive compounds, antioxidants, and essential nutrients, ensuring prolonged effectiveness and enhanced physiological benefits.

### FIELD OF INVENTION

The present disclosure relates generally to functional beverages, and more particularly to formulation of an oxygen-enhanced adaptive functional beverage formulated with at least one of advanced liposomal encapsulation, plant-based microencapsulation, and microbubble oxygenation technologies to enhance bioavailability and stability, which optimizes cellular metabolism, mitochondrial function, and oxygen utilization, boosting energy levels, cognitive function, and oxidative stress resistance, thereby supporting healthy aging, athletic performance, and high-altitude adaptation.

### BACKGROUND

Functional beverages have gained significant popularity as consumers seek convenient and scientifically formulated drinks to support energy, cognitive function, athletic recovery, and longevity. The demand for beverages that enhance cellular metabolism, mitochondrial efficiency, and oxygen utilization has surged, particularly among athletes, professionals, and individuals undergoing hyperbaric oxygen therapy (HBOT). However, existing functional beverages often rely on high-caffeine, high-sugar, or stimulant-based formulations, which fail to provide long-term metabolic and mitochondrial benefits.

Despite the growing demand, conventional functional beverages face several key challenges. Most energy drinks rely on caffeine, taurine, or guarana, which provide a temporary boost but do not enhance cellular energy production at the mitochondrial level. Ingredients like Coenzyme Q10 (CoQ10), Omega-3, and PQQ have poor solubility and absorption in standard beverage formulations, reducing their effectiveness. High-intensity exercise and HBOT create oxidative stress, requiring antioxidants such as Astaxanthin and Resveratrol to neutralize free radicals.

However, traditional beverages do not adequately address oxidative damage at the cellular level. Many functional drinks use high carbonation, leading to gastric distress and bloating, making them unsuitable for athletes, wellness consumers, and oxygen therapy patients. HBOT increases oxygen levels in the blood, but without proper mitochondrial support, the excess oxygen can contribute to oxidative stress instead of being efficiently utilized. No existing functional beverage is designed to enhance oxygen utilization under high-pressure conditions.

To address these issues, several technologies and ingredient formulations have been explored. Nanoemulsion Technology is used to enhance the absorption of fat-soluble nutrients like Omega-3 and CoQ10.Plant-based microencapsulation protects bioactive ingredients from oxidation and improves shelf stability. Electrolyte-infused beverages are used in sports drinks to replenish lost minerals and enhance hydration. Green tea extracts (EGCG) and Vitamin C are commonly used to reduce oxidative stress. Some formulations include herbal extracts (e.g., Ginseng, Rhodiola) to reduce fatigue and improve endurance.

Despite advancements in beverage technology, current solutions still fall short in addressing the comprehensive needs of metabolic health, oxygen optimization, and mitochondrial efficiency. Many beverages use these technologies separately, rather than in an integrated system to maximize absorption, stability, and effectiveness. While sports drinks replenish electrolytes, they do not enhance cellular energy metabolism or oxygen utilization, making them insufficient for HBOT users and endurance athletes.

Standard antioxidant drinks reduce oxidative stress but do not actively enhance oxygen efficiency at the cellular level. High-carbonation beverages cause bloating and are unsuitable for those with sensitive digestion or undergoing hyperbaric oxygen therapy. Many functional beverages fail to consider the synergistic effects of NMN, PQQ, CoQ10, and NAD+ in boosting mitochondrial function and cellular repair.

Therefore, there is a need for a functional beverage that supports long-term cellular energy metabolism without relying on stimulants. There is also a need for a functional beverage that uses optimized nanoemulsion and plant-based microencapsulation to enhance nutrient bioavailability. There is also a need for a functional beverage that combines oxygen-enhancing active factors with antioxidants to support hyperbaric oxygen therapy (HBOT) users and high-performance individuals. There is also a need for a functional beverage that eliminates digestive discomfort by offering both soft-carbonation and non-carbonated options. There is also a need for a functional beverage that provides a scientifically balanced formulation for metabolic health, endurance, cognitive function, and longevity.

### SUMMARY OF THE INVENTION

The following presents a simplified summary of one or more embodiments of the present disclosure to provide a basic understanding of such embodiments. This summary is not an extensive overview of all contemplated embodiments and is intended to neither identify key nor critical elements of all embodiments, nor delineate the scope of any or all embodiments.

The present disclosure, in one or more embodiments, relates to an oxygen-enhanced adaptive functional beverage formulated with at least one of advanced liposomal encapsulation, plant-based microencapsulation, and microbubble oxygenation technologies to enhance bioavailability and stability, which optimizes cellular metabolism, mitochondrial function, and oxygen utilization, boosting energy levels, cognitive function, and oxidative stress resistance, thereby supporting healthy aging, athletic performance, and high-altitude adaptation.

An embodiment of the first aspect, a method for preparing an oxygen-enhanced adaptive functional beverage. The oxygen-enhanced adaptive functional beverage comprises one or more pyridine nucleotides in an amount of about 9% to 13% by weight of a total beverage composition, one or more quinone-based coenzymes in an amount of about 12% to 16% by weight of the total beverage composition, one or more neurocalming compounds in an amount of about 8% to 12% by weight of the total beverage composition, one or more neurotransmitter precursors in an amount of about 9% to 13% by weight of the total beverage composition, a high-oxygen active compound in an amount of about 12% to 16% by weight of the total beverage composition, an olive extract in an amount of about 4% to 7% by weight of the total beverage composition, a rhodiola extract in an amount of about 4% to 9% by weight of the total beverage composition, an oxygen-rich hydration complex in an amount of about 12% to 18% by weight of the total beverage composition, and one or more additives in an amount of about 20% to 30% by weight of the total beverage composition.

In one embodiment, the pyridine nucleotides comprise at least one of nicotinamide adenine dinucleotide (NAD+), and nicotinamide mononucleotide (NMN). The quinone-based coenzymes comprise at least one of pyrroloquinoline quinone (PQQ), and coenzyme Q10 (CoQ10). The neurocalming compounds comprise at least one of gamma-aminobutyric acid (GABA), and L-theanine. The more neurotransmitter precursors comprise at least one of 5-Hydroxytryptophan (5-HTP), and alpha-glycerophosphocholine (Alpha-GPC). The high-oxygen active compound comprises at least one of dissolved molecular oxygen, rhodiola extract, hydroxytyrosol, an oxygen-rich hydration complex, and active iron/copper enzymes. The olive extract comprises hydroxytyrosol. The oxygen-rich hydration complex comprises at least one of magnesium, silica, and zinc.

In one embodiment of the invention, a method for preparing the oxygen-enhanced adaptive functional beverage is disclosed. At one step, the pyridine nucleotides are dissolved under precisely controlled temperature and pH conditions at least 6.5 to 7.2 to obtain a solution. The solution is subjected to a plant-based microencapsulation process to yield a microencapsulated mixture. At another step, the quinone-based coenzymes are subjected through a liposomal encapsulation process to obtain a liposomal formulation. The PQQ and the CoQ10 are processed using the liposomal encapsulation process, with droplet sizes ranging from 30-50 nm, to improve solubility and bioavailability.

At another step, the neurocalming compounds and the neurotransmitter precursors are subjected through an ultrasonic dispersion process to obtain a uniformly dispersed mixture. The neurocalming compounds comprise at least one of gamma-aminobutyric acid (GABA), and L-theanine. The neurotransmitter precursors comprise at least one of 5-Hydroxytryptophan (5-HTP), and alpha-glycerophosphocholine (Alpha-GPC). The ultrasonic dispersion process is carried out at a temperature below 10° C. and with a pH level between 5.8 and 6.8 to prevent degradation of the neurocalming compounds and the neurotransmitter precursors.

At another step, hydrogen-enriched water and oxygen-enriched water are prepared for infusion into a mixture of the microencapsulated mixture, the liposomal formulation, and the uniformly dispersed mixture to obtain an intermediate mixture. The hydrogen-rich water is produced via magnesium-based electrolysis or standard electrolysis-based hydrogen enrichment to achieve a hydrogen concentration of 1.0-2.5 ppm. The oxygen-enriched water is produced by infusing water with high levels of oxygen using microbubble oxygenation process to achieve a dissolved oxygen concentration of at least 30 ppm.

At another step, a high-oxygen active compound is integrated into the intermediate mixture through microbubble oxygenation process to obtain the oxygen-enhanced adaptive functional beverage. At another step, the oxygen-enhanced adaptive functional beverage is homogenized at temperatures below 10° C. using high-pressure homogenization to micronize active ingredients of the oxygen-enhanced adaptive functional beverage to 100-300 nm for improved cellular uptake. Further, at another step, the oxygen-enhanced adaptive functional beverage is filled under cold sterile conditions, at temperatures varies between 4° C. and 8° C., to prevent oxidation and preserve potency. The oxygen-enhanced adaptive functional beverage is packaged in antioxidant-coated aluminum cans or glass bottles, sealed with nitrogen gas, to prevent oxidation and extend shelf life.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

The accompanying drawings, which are incorporated in and constitute a part of the specification, illustrate an embodiment of the invention, and, together with the description, explain the principles of the invention.

FIG. 1 illustrates a flowchart for formulation of an oxygen-enhanced adaptive functional beverage for anti-aging, in accordance with embodiments of the invention.

### DETAILED DESCRIPTION

Reference will now be made in detail to the present preferred embodiments of the invention, examples of which are illustrated in the accompanying drawings. Wherever possible, the same reference numerals are used in the drawings and the description to refer to the same or like parts.

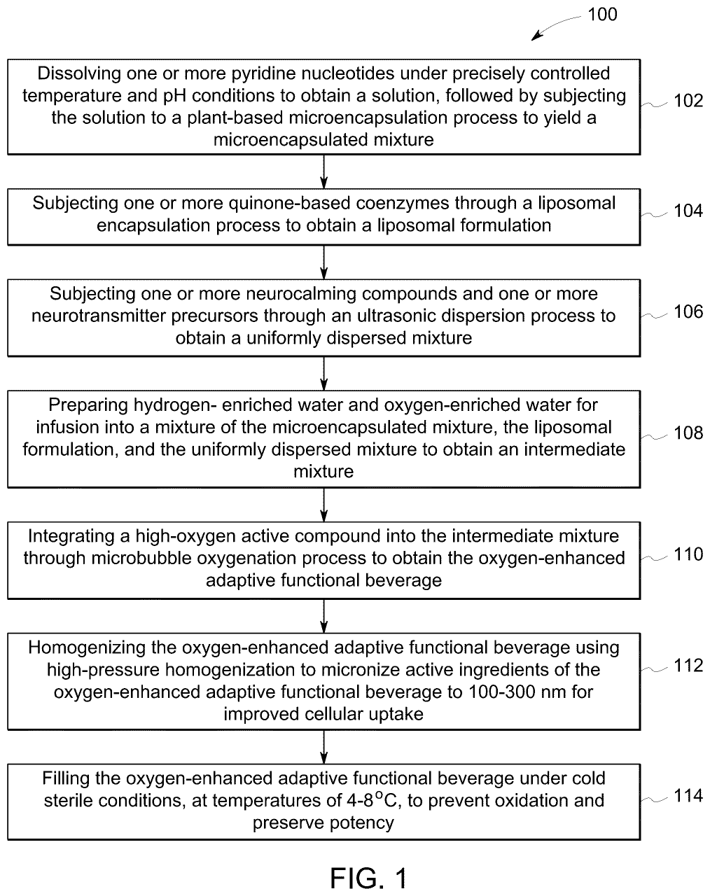

FIG. 1 refers to a flowchart 100 of a method for preparing an oxygen-enhanced adaptive functional beverage. The oxygen-enhanced adaptive functional beverage comprises one or more pyridine nucleotides in an amount of about 9% to 13% by weight of a total beverage composition, one or more quinone-based coenzymes in an amount of about 12% to 16% by weight of the total beverage composition, one or more neurocalming compounds in an amount of about 8% to 12% by weight of the total beverage composition, one or more neurotransmitter precursors in an amount of about 9% to 13% by weight of the total beverage composition, a high-oxygen active compound in an amount of about 12% to 16% by weight of the total beverage composition, an olive extract in an amount of about 4% to 7% by weight of the total beverage composition, a rhodiola extract in an amount of about 4% to 9% by weight of the total beverage composition, an oxygen-rich hydration complex in an amount of about 12% to 18% by weight of the total beverage composition, and one or more additives in an amount of about 20% to 30% by weight of the total beverage composition.

In one embodiment, the pyridine nucleotides comprise at least one of nicotinamide adenine dinucleotide (NAD+), and nicotinamide mononucleotide (NMN). The quinone-based coenzymes comprise at least one of pyrroloquinoline quinone (PQQ), and coenzyme Q10 (CoQ10). The neurocalming compounds comprise at least one of gamma-aminobutyric acid (GABA), and L-theanine. The more neurotransmitter precursors comprise at least one of 5-Hydroxytryptophan (5-HTP), and alpha-glycerophosphocholine (Alpha-GPC). The high-oxygen active compound comprises at least one of dissolved molecular oxygen, rhodiola extract, hydroxytyrosol, an oxygen-rich hydration complex, and active iron/copper enzymes. The olive extract comprises hydroxytyrosol. The oxygen-rich hydration complex comprises naturally oxygen-enriched minerals selected from the group consisting of magnesium, silica, and zinc, for optimizing oxygen transport efficiency. The additives comprise, but are not limited to, water, sweeteners, and other similar components.

At step 102, the pyridine nucleotides are dissolved under precisely controlled temperature and pH conditions at least 6.5 to 7.2 to obtain a solution. The solution is subjected to a plant-based microencapsulation process to yield a microencapsulated mixture. In one embodiment, the plant-based microencapsulation process employs plant-derived encapsulating agents, such as, but not limited to, acacia gum, maltodextrin, or whey protein, to enhance stability of the pyridine nucleotides. Acacia gum, a natural polysaccharide, functions as an emulsifier and stabilizer, providing a protective film that prevents degradation. Maltodextrin, a carbohydrate-based encapsulant, improves water solubility and controls the release of the core material. Whey protein, though animal-derived, can also be used as a stabilizing agent due to its excellent film-forming and emulsifying properties. The plant-based microencapsulation process aids to protect the encapsulated pyridine nucleotides from environmental factors such as moisture, oxygen, and heat, thereby prolonging shelf life and maintaining the functional integrity of the encapsulated pyridine nucleotides.

At step 104, the quinone-based coenzymes are subjected through a liposomal encapsulation process to obtain a liposomal formulation. The PQQ and the CoQ10 are processed using the liposomal encapsulation process, with droplet sizes ranging from 30-50 nm, to improve solubility and bioavailability. In one embodiment, use liposomal encapsulation process (phospholipid-based encapsulation) to improve the bioavailability of PQQ, CoQ10, and NMN.

At step 106, the neurocalming compounds and the neurotransmitter precursors are subjected through an ultrasonic dispersion process to obtain a uniformly dispersed mixture for enhanced ingredient distribution. The neurocalming compounds comprise at least one of gamma-aminobutyric acid (GABA), and L-theanine. The neurotransmitter precursors comprise at least one of 5-Hydroxytryptophan (5-HTP), and alpha-glycerophosphocholine (Alpha-GPC). The ultrasonic dispersion process is carried out at a temperature below 10° C. and with a pH of 5.8-6.8 to prevent degradation of the neurocalming compounds and the neurotransmitter precursors.

At step 108, hydrogen-enriched water and oxygen-enriched water are prepared for infusion into a mixture of the microencapsulated mixture, the liposomal formulation, and the uniformly dispersed mixture to obtain an intermediate mixture. The hydrogen-rich water is produced via magnesium-based electrolysis or standard electrolysis-based hydrogen enrichment to achieve a hydrogen concentration of 1.0-2.5 ppm. The standard electrolysis-based hydrogen enrichment process continuously generates hydrogen-enriched water through magnesium-based electrolysis. In this method, a magnesium electrode is immersed in water and subjected to an electrochemical reaction that releases hydrogen gas. When an electric current passes through the mixture of the microencapsulated mixture, the liposomal formulation, and the uniformly dispersed mixture, the magnesium undergoes oxidation, producing magnesium ions (Mg²⁺) while facilitating the reduction of water at the cathode to generate dissolved molecular hydrogen (H₂). The oxygen-enriched water is produced by infusing water with high levels of oxygen using microbubble oxygenation process to achieve a dissolved oxygen concentration of at least 30 ppm. In one embodiment, the intermediate mixture is infused with hydrogen and oxygen microbubbles, generated through microbubble oxygenation process, to improve oxygen uptake efficiency.

At step 110, a high-oxygen active compound is integrated into the intermediate mixture through microbubble oxygenation process to obtain the oxygen-enhanced adaptive functional beverage. In one embodiment, the high-oxygen active compound comprises dissolved molecular oxygen stabilized via the microbubble oxygenation process to enhance oxygen bioavailability.

At step 112, the oxygen-enhanced adaptive functional beverage is homogenized at temperatures below 10° C. using high-pressure homogenization to micronize active ingredients of the oxygen-enhanced adaptive functional beverage to 100-300 nm for improved cellular uptake.

At step 114, the oxygen-enhanced adaptive functional beverage is filled under cold sterile conditions, at temperatures varies between 4° C. and 8° C., to prevent oxidation and preserve potency. The oxygen-enhanced adaptive functional beverage is packaged in antioxidant-coated aluminum cans or glass bottles, sealed with nitrogen gas, to prevent oxidation and extend shelf life.

In a preferred embodiment, every 500 ml of the oxygen-enhanced adaptive functional beverage comprises at 250-500 mg of NMN, 50-100 mg of NAD+, 10-20 mg of PQQ, 30-100 mg of CoQ10, 50-200 mg of GABA, 100-300 mg of L-Theanine, 50-200 mg of 5-HTP precursor, and 100-250 mg of Alpha-GPC.

In another embodiment, the oxygen-enhanced adaptive functional beverage further comprises at least one antioxidant selected from astaxanthin neurotransmitter modulation blend that includes, resveratrol, vitamin C, vitamin E, and epigallocatechin gallate (EGCG). In another embodiment, the oxygen-enhanced adaptive functional beverage further comprises at least one electrolyte selected from Sodium (Na), Potassium (K), Magnesium (Mg), and Calcium (Ca). In another embodiment, the oxygen-enhanced adaptive functional beverage further comprises at least one natural sweetener selected from Erythritol and Stevia.

In one embodiment, the oxygen-enhanced adaptive functional beverage is an oxygen-enhanced adaptive functional beverage formulated with at least one of advanced liposomal encapsulation, plant-based microencapsulation, and microbubble oxygenation technologies to enhance bioavailability and stability, which optimizes cellular metabolism, mitochondrial function, and oxygen utilization, boosting energy levels, cognitive function, and oxidative stress resistance, thereby supporting healthy aging, athletic performance, and high-altitude adaptation.

In one embodiment herein, the oxygen-enhanced adaptive functional beverage with NMN, PQQ, CoQ10, and NAD+ to boost mitochondrial function and ATP synthesis, ensure long-term, sustained energy without relying on stimulants like caffeine or taurine. The oxygen-enhanced adaptive functional beverage with the high-oxygen active compound, configured to enhance blood oxygenation and cellular metabolism. Thereby, making the oxygen-enhanced adaptive functional beverage suitable for hyperbaric oxygen therapy (HBOT) users, endurance athletes, and recovery patients.

In one embodiment, daily intake of 500 mg NMN for 90 days showed no impact on blood sugar, liver function, or kidney function, confirming long-term safety. A 90-day trial on PQQ & CoQ10 (40 mg & 300 mg/day) reported no adverse effects. A 12-week intake of 200 mg GABA & 100 mg 5-HTP improved relaxation with no neurological damage and 30% reduction in anxiety scores.

In one embodiment herein, the method for formulating the oxygen-enhanced adaptive functional beverage utilizes nanoemulsion and plant-based microencapsulation processes to increase the absorption rate of fat-soluble and water-soluble bioactives (e.g., Omega-3, PQQ, Astaxanthin, Resveratrol), improving effectiveness by 3-5 times. The oxygen-enhanced adaptive functional beverage combines Astaxanthin, Resveratrol, EGCG (Green Tea Extract), and Vitamin C to neutralize free radicals, reducing oxidative stress and inflammation while supporting cardiovascular and skin health. In one embodiment herein, the oxygen-enhanced adaptive functional beverage is suitable for athletes, professionals, and longevity-focused consumers.

Further, the oxygen-enhanced adaptive functional beverage infused with GABA, L-Theanine, Alpha-GPC, and 5-HTP (Tryptophan precursor) to improve mental clarity, focus, stress reduction, and mood stabilization, making it ideal for high-performance professionals and individuals with cognitive demands. Use B-cyclodextrin or a-cyclodextrin to improve solubility and protect sensitive bio-actives of the oxygen-enhanced adaptive functional beverage without nanotechnology. Cyclodextrins, cyclic oligosaccharides with a hydrophobic inner cavity and a hydrophilic outer surface, form host-guest inclusion complexes with bioactive compounds. This encapsulation mechanism shields the bioactives from environmental degradation caused by moisture, oxygen, light, or heat while enhancing their water solubility and controlled release properties. B-Cyclodextrin, with a moderate-sized cavity, is well-suited for encapsulating a broad range of hydrophobic bioactives, while a-cyclodextrin, with a smaller cavity, is particularly effective for stabilizing smaller hydrophobic molecules.

In one embodiment herein, the oxygen-enhanced adaptive functional beverage comprises a balanced blend of sodium, potassium, magnesium, and calcium to maintain optimal hydration, nerve signaling, and muscle recovery, ensuring peak athletic performance and endurance. The oxygen-enhanced adaptive functional beverage comprises low-carbonation (soft) and non-carbonated (still) oxygen-enhanced adaptive functional beverage, eliminating issues like bloating and acidity that are common with traditional high-carbonation energy drinks.

In one embodiment herein, the oxygen-enhanced adaptive functional beverage is specifically designed to support individuals undergoing oxygen therapy, enhancing oxygen efficiency and reducing oxidative stress during treatment. In one embodiment herein, the oxygen-enhanced adaptive functional beverage is developed using clinically studied ingredients that comply with GRAS (Generally Recognized as Safe) regulations, ensuring safety and efficacy for daily consumption.

In one embodiment herein, the oxygen-enhanced adaptive functional beverage stands out as a next-generation functional beverage, bridging the gap between sports nutrition, longevity science, and metabolic health, offering a first-in-market solution for oxygen therapy users, high-performance individuals, and wellness-conscious consumers.

In one embodiment herein, the oxygen-enhanced adaptive functional beverage is formulated using nano emulsification, plant-based microencapsulation, and/or microbubble oxygenation technology to enhance ingredient bioavailability, stability, and absorption in high-oxygen environments. The oxygen-enhanced adaptive functional beverage integrates mitochondrial boosters, oxygen adaptation factors, and advanced bioavailability technologies in a single, optimized formulation.

In one embodiment herein, the oxygen-enhanced adaptive functional beverage is a low-carbonation or non-carbonated health beverage formulated with bioactive ingredients designed to enhance cellular metabolism, optimize mitochondrial function, and improve oxygen utilization. The oxygen-enhanced adaptive functional beverage integrates nanoemulsion, plant-based microencapsulation, and microbubble technologies to maximize nutrient bioavailability, ensuring efficient absorption of key compounds such as Nicotinamide Mononucleotide (NMN), Pyrroloquinoline Quinone (PQQ), Coenzyme Q10 (CoQ10), NAD+, Omega-3, Astaxanthin, and neurotransmitter modulators.

In one embodiment herein, the oxygen-enhanced adaptive functional beverage is applicable to athletic performance enhancement, cognitive support, anti-aging and longevity, and hyperbaric oxygen therapy (HBOT) supplementation, offering a scientifically optimized formulation for sustained energy, recovery, and metabolic health. Additionally, the oxygen-enhanced adaptive functional beverage is designed to minimize digestive discomfort by offering both soft-carbonation and non-carbonated versions, making it suitable for a broad range of consumers, including athletes, high-performance professionals, longevity-focused individuals, and medical recovery patients.

In one embodiment herein, the plant-based microencapsulation process extends the stability and shelf life of the oxygen-enhanced adaptive functional beverage by at least 12 months under standard storage conditions. The liposomal encapsulation process increases the bioavailability of fat-soluble ingredients of the oxygen-enhanced adaptive functional beverage by at least 3-5 times compared to conventional formulations.

TABLE 1

| | Energy | Cognitive | Physical | Sleep |
|---|---|---|---|---|
| Consumption | Boost | Clarity | Endurance | Disturbance |
| Method | (1-10) | (1-10) | (1-10) | (1-10) |
| NMN 300 mg + | 9 | 9 | 8 | 7 (Higher |
| PQQ 20 mg | | | | likelihood |
| (on an empty | | | | of sleep |
| stomach) | | | | disruption) |
| Post-meal mixed | 8 | 9 | 9 | 3 (Minimal |
| formulation | | | | sleep impact) |
| Optimized beverage | 7 | 8 | 8 | 2 (No sleep |
| (reduced PQQ/NMN | | | | impact) |
| content) | | | | |

Table 1 is derived from personal experimentation, serves as preliminary user experience evidence supporting the the oxygen-enhanced adaptive functional beverage.

In another embodiment, NAD+ shows effectiveness in various mouse models of human diseases. NAD+ is further effective in treating hypercholesterolemia, lowering LDL, and significantly raising HDL levels. NAD+ precursors are available in different forms, including extended-release formulations to reduce side effects like flushing. The combination of NAM and NAD+ has potential for multiple health benefits. NAD+ increases vitality, reducing mortality risks, and extending a healthy lifespan. NAM and NAD+ enhance mitochondrial function and energy metabolism. In another embodiment, NMN supplementation restores NAD+ levels, enhances mitochondrial function, and slows age-related decline.

In another embodiment, PQQ supplementation promotes mitochondrial biogenesis and boosts oxidative metabolism, resulting in increased energy production. Over an 8-week period, it also reduces oxidative stress and enhances cognitive function. PQQ acts as a potent antioxidant, reducing oxidative stress by neutralizing harmful free radicals and lowering inflammation. By protecting neurons from oxidative damage, PQQ supplementation contributes to improved cognitive performance, including enhanced memory, attention, and mental clarity.

In another embodiment, CoQ10 plays a crucial role in cellular bioenergetics by facilitating electron transport within mitochondria, thereby optimizing ATP synthesis and supporting overall energy metabolism. CoQ10 enhances endurance by improving oxygen utilization and reducing muscle fatigue during physical exertion. CoQ10 supplementation helps mitigate oxidative stress by acting as a powerful antioxidant, protecting cells from free radical damage and reducing inflammation. Individuals supplementing with CoQ10 experience increased stamina, reduced exercise-induced fatigue, and enhanced recovery, making it an essential nutrient for athletes and those seeking improved cardiovascular and muscular health.

In another embodiment, Rhodiola enhances oxygen utilization, boosts endurance, and reduces physical fatigue, making it a valuable aid for athletic performance and overall stamina. Supplementation with Rhodiola has been shown to improve maximal oxygen uptake (VO2 max), confirming its effectiveness in enhancing endurance and optimizing aerobic capacity.

In another embodiment, hydroxytyrosol enhances mitochondrial function by supporting efficient energy production and protecting cells from oxidative stress. As a potent antioxidant, it reduces oxidative damage, minimizes cellular inflammation, and helps maintain mitochondrial integrity. Additionally, hydroxytyrosol plays a crucial role in improving metabolic health by promoting better lipid metabolism, enhancing insulin sensitivity, and supporting overall cellular function.

In one embodiment, the microbubble oxygenation significantly increases oxygen solubility in water by generating ultra-fine gas bubbles that enhance oxygen transfer efficiency. Maintain microbubble technology for oxygen infusion without nanoscale oxygen stabilization. This advanced oxygenation method has diverse applications in medical and sports recovery, where increased oxygen availability plays a crucial role in tissue repair and cellular metabolism. In sports and fitness, consuming microbubble oxygenated water has been shown to accelerate muscle recovery, reduce fatigue, and improve endurance by enhancing oxygen delivery to muscles and promoting faster lactate clearance. In medical applications, it supports wound healing, improves circulation, and aids in oxygen therapy for patients with conditions related to hypoxia or impaired oxygen utilization.

In some embodiments, method of consuming NMN and PQQ significantly influences their effects on energy levels, cognitive clarity, physical endurance, and sleep patterns. Taking 300 mg of NMN with 20 mg of PQQ on an empty stomach provides a strong energy boost by at least 90% and enhances cognitive function by at least 90% and physical endurance by at least 80%, but it also has a higher likelihood of sleep disturbances by at least 70%.

In some embodiments, a post-meal mixed formulation slightly reduces the energy boost by at least 80% while maintaining high cognitive clarity by at least 90% and improving physical endurance by at least 90%, with minimal sleep impact by at least 30%. An optimized beverage with reduced PQQ and NMN content provides a more moderate energy boost by at least 70%, good cognitive clarity by at least 80%, and stable physical endurance by at least 80%, while eliminating sleep disturbances by at least 20%. These variations highlight the importance of dosing strategy and timing in optimizing benefits while minimizing side effects.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principles of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

It will readily be apparent that numerous modifications and alterations can be made to the processes described in the foregoing examples without departing from the principles underlying the invention, and all such modifications and alterations are intended to be embraced by this application.

The claimed invention is:

1. An oxygen-enhanced adaptive functional beverage, comprising:
one or more pyridine nucleotides in an amount of about 9% to 13% by weight of a total beverage composition;
one or more quinone-based coenzymes in an amount of about 12% to 16% by weight of the total beverage composition;
one or more neurocalming compounds in an amount of about 8% to 12% by weight of the total beverage composition;
one or more neurotransmitter precursors in an amount of about 9% to 13% by weight of the total beverage composition;
a high-oxygen active compound in an amount of about 12% to 16% by weight of the total beverage composition;
an olive extract in an amount of about 4% to 7% by weight of the total beverage composition;
a rhodiola extract in an amount of about 4% to 9% by weight of the total beverage composition;
an oxygen-rich hydration complex in an amount of about 12% to 18% by weight of the total beverage composition; and
one or more additives in an amount of about 20% to 30% by weight of the total beverage composition,
wherein the oxygen-enhanced adaptive functional beverage is formulated to enhance mitochondrial function, oxygen utilization, and antioxidant defense.

2. The oxygen-enhanced adaptive functional beverage of claim 1, wherein the one or more pyridine nucleotidescomprise at least one of nicotinamide adenine dinucleotide (NAD+), and nicotinamide mononucleotide (NMN).

3. The oxygen-enhanced adaptive functional beverage of claim 1, wherein the one or more quinone-based coenzymes comprise at least one of pyrroloquinoline quinone (PQQ), and coenzyme Q10 (CoQ10).

4. The oxygen-enhanced adaptive functional beverage of claim 1, wherein the one or more neurocalming compounds comprise at least one of gamma-aminobutyric acid (GABA), and L-theanine.

5. The oxygen-enhanced adaptive functional beverage of claim 1, wherein the one or more neurotransmitter precursors comprise at least one of 5-Hydroxytryptophan (5-HTP), and alpha-glycerophosphocholine (Alpha-GPC).

6. The oxygen-enhanced adaptive functional beverage of claim 1, wherein the high-oxygen active compound comprises at least one of dissolved molecular oxygen, rhodiola extract, hydroxytyrosol, an oxygen-rich hydration complex, and active iron/copper enzymes.

7. The oxygen-enhanced adaptive functional beverage of claim 1, wherein the olive extract comprises hydroxytyrosol.

8. The oxygen-enhanced adaptive functional beverage of claim 1, wherein the oxygen-rich hydration complex comprises at least one of magnesium, silica, and zinc.

9. The oxygen-enhanced adaptive functional beverage of claim 1, wherein the one or more additives comprise at least one of water and sweeteners.

10. A method for preparing an oxygen-enhanced adaptive functional beverage, comprising:
dissolving one or more pyridine nucleotides under precisely controlled temperature and pH conditions to obtain a solution, followed by subjecting the solution to a plant-based microencapsulation process to yield a microencapsulated mixture;
subjecting one or more quinone-based coenzymes through a liposomal encapsulation process to obtain a liposomal formulation;
subjecting one or more neurocalming compounds and one or more neurotransmitter precursors through an ultrasonic dispersion process to obtain a uniformly dispersed mixture;
preparing hydrogen-enriched water and oxygen-enriched water for infusion into a mixture of the microencapsulated mixture, the liposomal formulation, and the uniformly dispersed mixture to obtain an intermediate mixture;
integrating a high-oxygen active compound into the intermediate mixture through microbubble oxygenation process to obtain the oxygen-enhanced adaptive functional beverage;
homogenizing the oxygen-enhanced adaptive functional beverage using high-pressure homogenization to micronize active ingredients of the oxygen-enhanced adaptive functional beverage to 100-300 nm for improved cellular uptake; and
filling the oxygen-enhanced adaptive functional beverage under cold sterile conditions, at temperatures varies between 4° C. and 8° C., to prevent oxidation and preserve potency.

11. The method of claim 10, wherein the one or more pyridine nucleotides comprise at least one of nicotinamide adenine dinucleotide (NAD+), and nicotinamide mononucleotide (NMN), wherein the NAD+ and the NMN are maintained at a pH level between 6.5 and 7.2.

12. The method of claim 10, wherein the one or more quinone-based coenzymes comprise at least one of pyrroloquinoline quinone (PQQ), and coenzyme Q10 (CoQ10).

13. The method of claim 10, wherein the one or more quinone-based coenzymes are processed using the liposomal encapsulation process, with droplet sizes ranging from 30 to 50 nm, to improve solubility and bioavailability.

14. The method of claim 10, wherein the ultrasonic dispersion process is carried out at a temperature below 10° C. and a pH level between 5.8 and 6.8 to prevent degradation of the one or more neurocalming compounds and the one or more neurotransmitter precursors.

15. The method of claim 10, wherein the hydrogen-rich water is produced through magnesium-based electrolysis or standard electrolysis-based hydrogen enrichment to achieve a hydrogen concentration of at least 1.0 to 2.5 ppm.

16. The method of claim 10, wherein the oxygen-enriched water is produced by infusing water with high levels of oxygen using microbubble oxygenation process to achieve a dissolved oxygen concentration of at least 30 ppm.

17. The method of claim 10, wherein the oxygen-enhanced adaptive functional beverage is homogenized at temperatures below 10° C.

18. The method of claim 10, wherein the oxygen-enhanced adaptive functional beverage is packaged in antioxidant-coated aluminum cans or glass bottles, sealed with nitrogen gas, to prevent oxidation and extend shelf life.

19. The method of claim 10, wherein the one or more additives comprise at least one of water and sweeteners.
